(19) United States
(12) Patent Application Publication
(10) Pub. No.: US 2021/0299412 A1
(43) Pub. Date: Sep. 30, 2021

(54) Guide Wires

(71) Applicant: ASAHI INTECC CO., LTD., Seto-Shi (JP)

(72) Inventors: Yasuhiro Kusano, Seto-shi (JP); Maiko Kataoka, Seto-shi (JP); Yu Shinohara, Seto-shi (JP)

(73) Assignee: ASAHI INTECC CO., LTD., Seto-Shi (JP)

(21) Appl. No.: 17/344,497

(22) Filed: Jun. 10, 2021

Related U.S. Application Data
(63) Continuation of application No. PCT/JP2019/004268, filed on Feb. 6, 2019.

(51) Int. Cl. A61M 25/09; A61M 25/00

(52) U.S. Cl. CPC A61M 25/09; A61M 25/0045; A61M 2025/09141; A61M 2025/09083; A61M 2025/09166

(57) ABSTRACT

A guide wire has a core shaft. The core shaft includes a body portion and a layered portion. The body portion contains a nickel-titanium-based alloy as a main component, the nickel-titanium-based alloy having a superelastic property. The layered portion includes an inner layer formed on a part of an outer peripheral face of the body portion and containing a nickel alloy as a main component, and an outer layer formed on the inner layer and containing a titanium oxide as a main component.

### CROSS-REFERENCE TO RELATED APPLICATION

This application is a continuation application of International Application No. PCT/JP2019/004268, filed Feb. 6, 2019, the contents of which are incorporated herein by reference in their entirety.

### FIELD OF INVENTION

The present invention relates generally to guide wires.

### BACKGROUND

When treating an intravascular occluded site (e.g., chronic total occlusion: CTO) or the like caused by progressive calcification, a guide wire for guiding therapeutic implements is inserted into a blood vessel prior to insertion of a therapeutic implement such as a balloon catheter.

Such a guide wire should have an excellent shape restorability such that it is restored to an original shape state from a bent shape state by bringing the distal end of the guide wire into contact with an occluded site in a blood vessel. In addition, since the guide wire needs to be directed in a specific blood vessel direction at a site where the blood vessel is branched, the guide wire should also be easy to form such that a distal end portion of the guide wire can be bent into a desired shape before insertion.

Among these properties, in relation to the ease of forming, for example, there is a known design in which a transverse section orthogonal to the longitudinal direction of a core shaft in the guide wire is formed into a flat shape according to JP2016-67385. Such a design is excellent in that the core shaft can be easily bent in a direction perpendicular to the flat direction.

For such a design, when pushing the guide wire forward in a branched blood vessel, the distal end portion of the guide wire should be directed in a particular blood vessel direction, and this operation is conducted by rotating a proximal end of the guide wire.

However, when the distal end portion of the core shaft in the guide wire is formed into a flat shape as described above, the aforementioned rotation of the distal end portion does not promptly follow the rotation of the proximal end, and the distal end portion of the core shaft that has been difficult to rotate at the initial stage of the rotation may suddenly begin to rotate in some cases. This phenomenon is called “repellence,” and when this repellence occurs, operability of the guide wire is decreased, and blood vessel selectivity of the guide wire is significantly reduced.

### SUMMARY

An object of the disclosed embodiments is to provide a guide wire in which the ease of forming the distal end portion can be enhanced while maintaining excellent overall shape restorability.

In order to achieve this object, a guide wire according to a disclosed embodiment includes a core shaft. The core shaft can have a body portion and a layered portion. The body portion can comprise a nickel-titanium-based alloy as a main component, the nickel-titanium-based alloy having a superelastic property. The layered portion can have an inner layer formed on a part of an outer peripheral face of the body portion, the inner layer comprising a nickel alloy as a main component; and an outer layer formed on the inner layer, the outer layer comprising a titanium oxide as a main component.

The terms “comprise” and any form thereof such as “comprises” and “comprising,” “have” and any form thereof such as “has” and “having,” “include” and any form thereof such as “includes” and “including,” and “contain” and any form thereof such as “contains” and “containing” are open-ended linking verbs. As a result, a device, like a guide wire, that “comprises,” “has,” “includes,” or “contains” one or more elements possesses those one or more elements, but is not limited to possessing only those elements. Likewise, a method that “comprises,” “has,” or “includes” one or more steps possesses those one or more steps, but is not limited to possessing only those one or more steps.

Any embodiment of any of the devices and methods can consist of or consist essentially of—rather than comprise/include/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

### BRIEF DESCRIPTION OF THE DRAWINGS

FIG. 1 is a schematic sectional view in an axial direction illustrating an embodiment of the present guide wires.

FIG. 2 is a schematic sectional view of the guide wire of FIG. 1 taken along line II-II.

FIG. 3A is a schematic sectional view in a transverse direction illustrating an embodiment of the present guide wires.

FIG. 3B is a schematic sectional view in a transverse direction illustrating an embodiment of the present guide wires.

FIG. 3C is a schematic sectional view in a transverse direction illustrating an embodiment of the present guide wires.

FIG. 4A is a schematic sectional view in an axial direction illustrating an embodiment of the present guide wires.

FIG. 4B is a schematic sectional view in an axial direction illustrating an embodiment of the present guide wires.

FIG. 5A illustrates an example of a mapping image of titanium (Ti) in a section including a layered portion of a core shaft.

FIG. 5B illustrates an example of a mapping image of nickel (Ni) in the section including the layered portion of the core shaft.

FIG. 5C is an SEM image presenting the mapping regions in FIG. 5A and FIG. 5B.

### DETAILED DESCRIPTION

The present guide wires can have a core shaft. The core shaft can include a body portion and a layered portion. The body portion can comprise a nickel-titanium-based alloy as a main component, the nickel-titanium-based alloy having a superelastic property. The layered portion can have an inner layer formed on a part of an outer peripheral face of the body portion and comprise a nickel alloy as a main component and an outer layer formed on the inner layer and comprise a titanium oxide as a main component.

In this specification, the “main component” means a component accounting for the largest mole fraction among the contained components at the relevant site. The “distal end portion” of the guide wire (core shaft) means a distal end site excluding a proximal end of an object, e.g. a small diameter portion, a tapered portion, and the like in the core shaft.

Hereinafter, embodiments of the present guide wires will be explained with reference to the figures, but the disclosed embodiments are not limited only to the embodiments described in the figures.

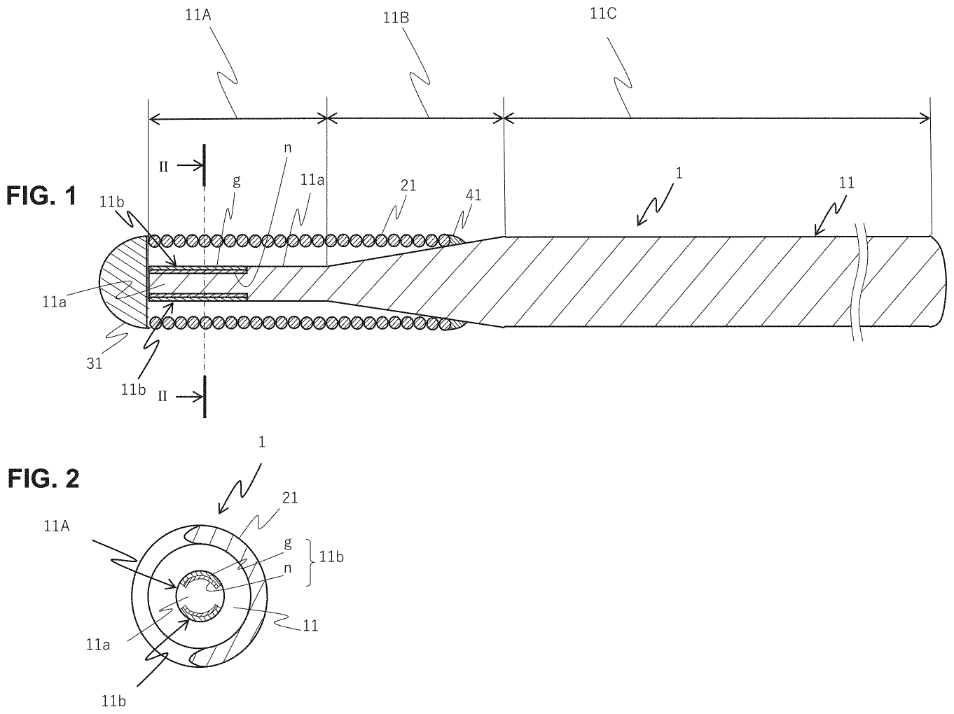

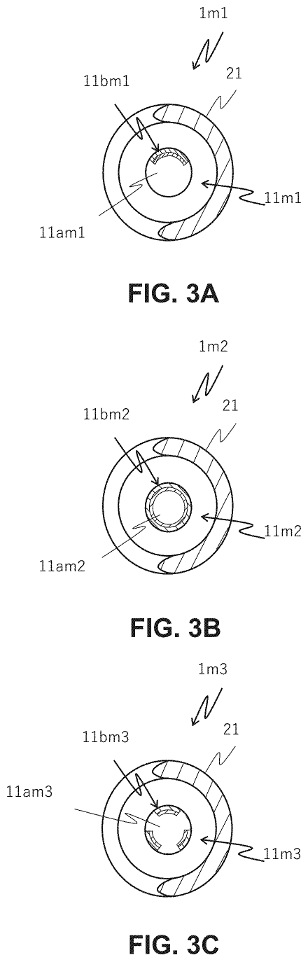

FIG. 1 is a schematic sectional view in an axial direction illustrating an embodiment 1 of the present guide wires and FIG. 2 is a schematic sectional view of guide wire 1 taken along line II-II in FIG. 1. As illustrated in FIG. 1 and FIG. 2, a guide wire 1 comprises a core shaft 11, a coil body 21, and a distal end fixing part 31.

The core shaft 11 can be a member constituting a central axis of the guide wire 1. The core shaft 11 can be formed, for example, such that the distal end portion thereof gradually decreases in diameter toward the distal end direction. In the guide wire 1, the core shaft 11 can comprise a small diameter portion 11A, a tapered portion 11B, and a large diameter portion 11C in this order from the distal end. When guide wire 1 extends in a straight line, the small diameter portion 11A can have a cylindrical shape, the large diameter portion 11C can have a cylindrical shape with an outer diameter larger than that of the small diameter portion 11A, and the tapered portion 11B can have a frustoconical shape that is continuous with the small diameter portion 11A and the large diameter portion 11C and gradually increases in diameter from the small diameter portion 11A to the large diameter portion 11C.

A total length of the core shaft 11 may be 1,800 to 3,000 mm, or 1,800 to 2,500 mm. A length in the axial direction of the small diameter portion 11A may be 0.5 to 50 mm, or 1 to 20 mm. A length in the axial direction of the tapered portion 11B may be 10 to 200 mm, or 20 to 150 mm. An outer diameter of the small diameter portion 11A may be 0.02 to 0.1 mm, or 0.03 to 0.07 mm. An outer diameter of the large diameter portion 11C may be 0.25 to 1 mm, or 0.35 to 0.46 mm.

In the embodiment of guide wire 1 shown, the total length of the core shaft 11 is 1,900 mm, the length in the axial direction of the small diameter portion 11A is 10 mm, the length in the axial direction of the tapered portion 11B is 100 mm, the outer diameter of the small diameter portion 11A is 0.090 mm, and the outer diameter of the large diameter portion 11C is 0.335 mm.

The core shaft 11 can include a body portion 11a and a layered portion 11b.

The body portion 11a refers to a site that can comprise a nickel-titanium-based alloy having a superelastic property as a main component in the core shaft 11.

Examples of the aforementioned nickel-titanium-based alloy include an Ni—Ti alloy (Ni=49 to 53 at % (atomic percentage)), an Ni—Ti—X alloy in which some of Ni atoms and/or Ti atoms in the Ni—Ti alloy are substituted with X atoms (e.g., X═Co, Fe, Mn, Cr, V, Al, Nb, W, or B, X=0.01 to 10 at %), an Ni—Ti—X alloy (X═Cu, Pb, or Zr, X=0.01 to 30 at %), and the like.

Among these alloys, the nickel-titanium alloy is preferable as the nickel-titanium-based alloy, and from the viewpoint of excellent superelastic property, an Ni—Ti alloy (Ni=49-53 at %) is more preferable. Thus, the core shaft 11 can acquire excellent shape restorability and high biocompatibility.

The layered portion 11b can have an inner layer n and an outer layer g. The inner layer n can be a site formed on a part on an outer peripheral face of the body portion 11a and can comprise a nickel alloy as a main component. This inner layer n can be adjacent to the body portion 11a via a thin boundary layer (also referred to as “inner layer-body portion boundary layer”), and a composition in the inner layer-body portion boundary layer can continuously vary from a composition of the body portion 11a to a composition of the inner layer n. The outer layer g can be a site formed on the inner layer n and comprise a titanium oxide (e.g. titanium (IV) oxide: TiO2, titanium( II) oxide: TiO, and/or the like) as a main component. An outer surface of this outer layer g can define at least a portion of an outer surface of the core shaft 11. In addition, the outer layer g can be adjacent to the inner layer n via a thin boundary layer (also referred to as “inner layer-outer layer boundary layer”), and a composition in the inner layer-outer layer boundary layer can continuously vary from the composition of the inner layer n to a composition of the outer layer g.

Examples of the aforementioned nickel alloy include an alloy like any of those described above for body portion 11a except without titanium atoms, and/or the like. Specific examples include an Ni—Cu alloy as the nickel alloy of inner layer n of layered portion 11b in a case of using a Ni—Ti—Cu alloy as a main component of body portion 11a, an Ni—Nb alloy as the nickel alloy of inner layer n of layered portion 11b in a case of using an Ni—Ti—Nb alloy as a main component of body portion 11a, and the like.

In the guide wire 1, the layered portion 11b can be only part of the small diameter portion 11A in the core shaft 11, and the body portion 1 la can be another part of core shaft 11 (site other than a part described above), including a part of the small diameter portion 11A, the tapered portion 11B, and the large diameter portion 11C.

The core shaft 11 can include two or more layered portions that are separated from each other, and it is possible that the two or more layered portions are arranged symmetrically with each other about a central axis of the core shaft 11 while sandwiching the body portion 11a therebetween in a cross-section of the core shaft 11 taken orthogonally to an axial direction of the core shaft 11. In the guide wire 1, as illustrated in FIG. 2, the small diameter portion 11A of the core shaft 11 includes two layered portions 11b and 11b that are separated from each other, and the two layered portions 11b and 11b are arranged symmetrically with each other about a central axis of the core shaft 11 while sandwiching the body portion 11a therebetween in a cross-section of the core shaft 11 taken orthogonally to an axial direction of the small diameter portion 11A.

In this way, the core shaft 11 includes two layered portions 11b and 11b that are arranged symmetrically with each other about the central axis of the core shaft 11 while sandwiching the body portion 11a therebetween, and thereby the core shaft 11 can be easily and reliably formed in a specific direction (in the guide wire 1, a direction from a central axis (body portion 11a) of the core shaft 11 toward the layered portion 11b).

The layered portions 11b can be formed by, for example, heating a surface of the core shaft 11 on which the layered portions 11b are formed by irradiating the surface with a laser light such as a YAG laser and a semiconductor laser (laser heating method), bringing the surface into direct contact with a high-temperature heat source (direct heating method), and the like. Among these methods, the laser heating method is preferable because it allows the layered portions 11b to accurately be formed only on a desired site in a short time. The region (area, depth) on which the layered portions 11b are formed is not particularly limited as long as the inner layer n is disposed on the outer peripheral face of the body portion 11a, and can be adjusted by appropriately selecting the heating conditions depending on a desired bent shape.

The coil body 21 is wound so as to cover at least a part of an outer periphery of the core shaft 11, and can comprise, for example, a single-thread coil or the like obtained by spirally winding one solid wire such that adjacent sections of the solid wire are in contact with each other.

A diameter of a wire constituting the coil body 21 may be 0.01 to 0.10 mm, or 0.01 to 0.08 mm. In the guide wire 1, a single-thread coil body 21 obtained by spirally winding a wire having a diameter of 0.06 mm is one example.

The wire constituting the coil body 21 can comprise, for example, a stainless steel such as SUS316; a superelastic alloy such as a Ni—Ti alloy; a radiopaque metal such as platinum and tungsten; or the like.

For example, the aforementioned coil body 21 can have a distal end fixed to the distal end fixing part 31 described below, and a proximal end fixed to the outer periphery of the core shaft 11 on a joint part 41. The coil body 21 can be fixed to the core shaft 11 with, for example, a brazing method or the like. Examples of a brazing material used in the aforementioned brazing method include a brazing metal such as an Sn—Pb alloy, an Pb—Ag alloy, an Sn—Ag alloy, and an Au—Sn alloy, and the like.

The distal end fixing part 31 can be a site where the distal end of the core shaft 11 and the distal end of the coil body 21 are fixed to each other. Specifically, in this distal end fixing part 31, for example, the distal end of the core shaft 11 and the distal end of the coil body 21 are integrally brazed. As for a shape of the distal end fixing part 31, so as not to damage an inner wall of a blood vessel when advancing the guide wire 1 in the blood vessel, for example, a brazing material can be used to form the distal end fixing part 31 into a hemispherical shape in which a distal end side portion of the distal end fixing part 31 is smoothly curved. Examples of the brazing material used for the distal end fixing part 31 include the same brazing materials as those described for the brazing method of the coil body 21 and the core shaft 11 as an example, and the like.

Next, an example of a usage mode of the guide wire 1 will be explained. First, a surgeon can bend the distal end portion of the guide wire 1, such as into a J-shape. The site to be bent in the guide wire 1 can be an axial region thereof that includes layered portion(s) 11b of the core shaft 11. In this region, the guide wire 1 can be bent into any desired shape as long as the bending direction is orientated from the central axis of the core shaft 11 toward one of the layered portion(s) 11b.

Subsequently, the distal end of the guide wire 1 having the bent distal end portion can be inserted into a blood vessel and then pushed toward a treatment site. At this time, for example, when the guide wire 1 reaches a branched site of the blood vessel, the distal end portion of the guide wire 1 can be rotated as necessary. The surgeon can rotate the proximal end of the guide wire 1 to rotate the distal end portion. After the guide wire 1 reaches a treatment site, an instrument such as a balloon catheter and a stent can be transported along the guide wire 1 to perform various treatments at the treatment site. After the treatment is completed, the guide wire 1 can be retracted in the blood vessel and drawn out from the body, and the series of procedures can be completed.

As described above, since the guide wire 1 has the aforementioned configuration, a local formability can be enhanced on the layered portion while maintaining excellent overall shape restorability by the superelastic property of the body portion 11a. It is inferred that this is because the superelastic property in the layered portion disappears due to the denaturation of the base material in association with the thermal action, and as a result, plastic deformation becomees possible. As a result, the guide wire 1 makes it possible to improve operability and perform procedures promptly and reliably.

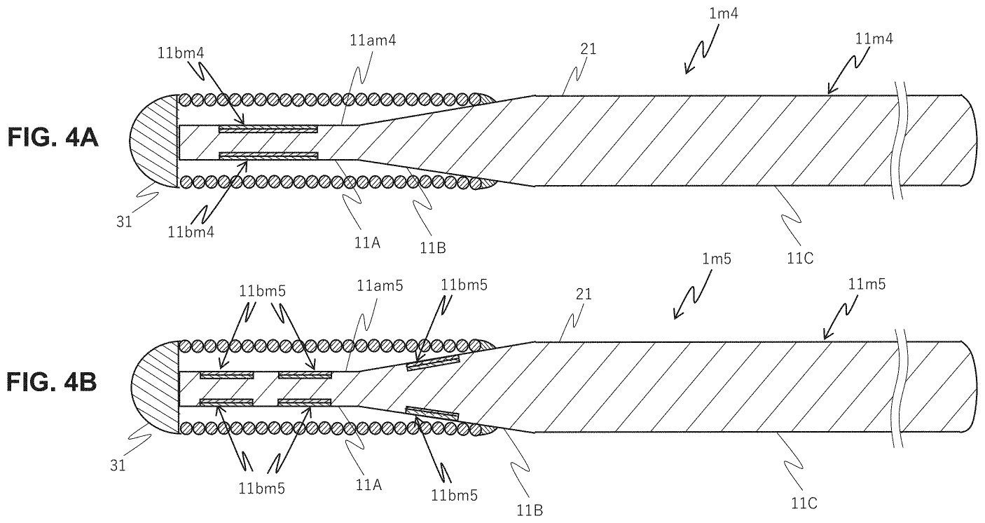

The disclosed embodiments are not limited to the configurations of the aforementioned embodiments. For example, while as described above the guide wire 1 can have two layered portions 11b and 11b of the core shaft 11 arranged symmetrically with each other about a central axis of the core shaft while sandwiching the body portion 11a therebetween, in other embodiments the guide wire may be, e.g., a guide wire 1m1 in which a layered portion 11bm1 is disposed only on one side region in an outer periphery of a body portion 11am1 in a cross-section of the core shaft 11m1 taken orthogonally to an axial direction of the core shaft 11am1, as illustrated in FIG. 3A. Also, this makes it possible to easily and reliably form the core shaft in a specific direction in the same manner as in the aforementioned embodiments. In addition, the arrangement of the layered portion in the cross-section taken orthogonally to the axial direction of the core shaft may be represented by not only the guide wire 1m1 in FIG. 3A but also by, e.g., a guide wire 1m2 in which a layered portion 11bm2 is arranged over an entire periphery of a body portion 11am2 in a cross-section of the core shaft 11m2 taken orthogonally to an axial direction of the core shaft 11m2 (see FIG. 3B), a guide wire 1m3 in which layered portions 11bm3 are independently disposed at three or more sites on an outer periphery of a body portion 11am3 in a cross-section of the core shaft 11m3 taken orthogonally to an axial direction of the core shaft 11m3 (see FIG. 3C), or the like.

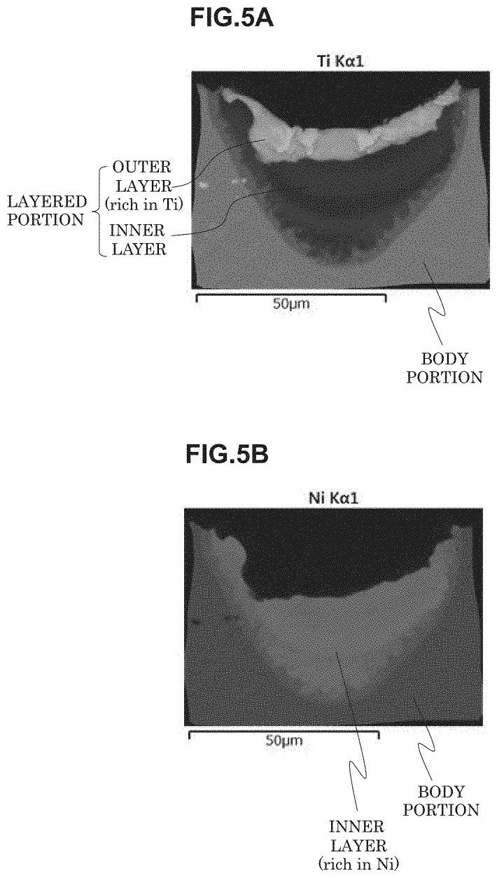

Additionally, with regard to the site of layered portions 11b in the axial direction of the core shaft 11, while as described above for the guide wire 1 the layered portions 11b can be continuous with the distal end fixing part 31 and are only a part of the small diameter portion 11A (no layered portion is formed on the tapered portion 11B and the large diameter portion 11C), in other embodiments the layered portions may be part of any site of the small diameter portion, the tapered portion, and the large diameter portion as long as the effects of the disclosed embodiments are not impaired. The layered portions may also be located at a plurality of sites in the axial direction of the core shaft. Examples include a guide wire 1m4 in which layered portions 11bm4 are disposed only on smaller diameter portion 11A of a core shaft 11m4 in an axial direction of a core shaft 11m4 but are disposed away from distal end fixing part 31 (see FIG. 4A), a guide wire 1m5 in which layered portions 11bm5 are independently disposed at a plurality of sites in an axial direction of a core shaft 11m5 (see FIG. 4B), and the like.

In addition, although as described above the guide wire 1 can have the small diameter portion 11A, the tapered portion 11B, and the large diameter portion 11C, in other embodiments the guide wire may be a guide wire including no small diameter portion and/or tapered portion, or a guide wire including a core shaft having a distal end portion with another shape.

Also, although as described above the guide wire 1 can include the coil body 21 and the distal end fixing part 31, in other embodiments the guide wire may be a guide wire including a coil body and a distal end fixing part that have other shapes, or a guide wire including no coil body and/or distal end fixing part.

### EXAMPLES

Hereinafter, the disclosed embodiments will be explained with reference to the following Examples, but the disclosed embodiments are not limited to the Examples.

### Example 1—Production of Core Shaft

Using an Ni—Ti alloy (Ni=51 at %) as a base material and by centerless polishing, a core shaft having a total length of 1,900 mm was formed, which had a small diameter portion (cylindrical shape, length in the axial direction: 10 mm, outer diameter: 0.090 mm), a tapered portion (frustoconical shape, length in the axial direction: 100 mm), and a large diameter portion (cylindrical shape, outer diameter: 0.335 mm) in this order from the distal end.

Subsequently, using the obtained core shaft, a laser light (fiber laser) was emitted onto two surface regions that each spanned 5 mm from the distal end toward the proximal end of the small diameter portion in this core shaft. The surface regions were opposite to each other with respect to the central axis of the core shaft. A core shaft including two layered portions separated from each other and arranged symmetrically with each other about the central axis of the core shaft while sandwiching a body portion therebetween in a cross-section of the core shaft taken orthogonally to an axial direction of the core shaft was thus obtained.

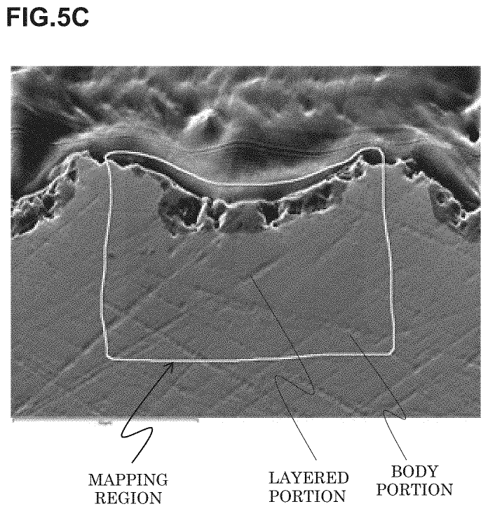

FIG. 5A and FIG. 5B are mapping images of titanium (Ti) and nickel (Ni), respectively, in a section of the core shaft including the layered portion, which was analyzed using an energy dispersive X-ray spectrometer (EDX, Energy dispersive X-ray Spectrometry, Model: AZtec Energy Advanced X-Max50, manufactured by Oxford Instruments) annexed to a field emission type scanning electron microscope (Model: SU-70, manufactured by Hitachi High-Tech Corporation). FIG. 5C is a SEM (scanning electron microscope) image of the aforementioned section (the mapping area is inside the white line in FIG. 5C). As can be seen from these mapping images, in Example 1, a part of the Ni—Ti alloy used as the base material disappeared by irradiating the surface of the core shaft with the laser light, and a layered portion composed of an outer layer containing Ti atoms and an inner layer without Ti atoms was formed on a part of the outer peripheral face of the body portion.

### Example 2-Production of Guide Wire

A single-thread coil body (material: platinum and stainless steel, wire diameter: 0.06 mm, coil outer diameter: 0.345 mm, length: 110 mm) previously wound was used, and the core shaft produced in Example 1 was inserted into a central hole of the coil body. Then, the distal end of the coil body and the distal end of the core shaft were integrally brazed using a brazing material, to form a hemispherical distal end fixing part, and the proximal end of the coil body was brazed to the outer peripheral face of the tapered portion of the core shaft to form a joint part, so that a guide wire of Example 2 was obtained.

1. A guide wire having a core shaft, wherein:
the core shaft comprises a body portion and one or more layered portions;
the body portion comprises a superelastic nickel-titanium-based alloy as a main component; and
each of the layered portion(s) has:
an inner layer formed on a part of an outer peripheral face of the body portion, the inner layer comprising a nickel alloy as a main component; and
an outer layer formed on the inner layer and comprising a titanium oxide as a main component.

2. The guide wire of claim 1, wherein:
the one or more layered portions comprise two or more layered portions that are separated from each other; and
the two or more layered portions are arranged symmetrically with each other about a central axis of the core shaft while sandwiching the body portion therebetween in a cross-section of the core shaft taken orthogonally to an axial direction of the core shaft.

3. The guide wire of claim 1, wherein a cross-section of the core shaft taken orthogonally to an axial direction of the core shaft includes a single one of the layered portion(s) disposed along less than half of an outer periphery of the body portion in the cross-section.

4. The guide wire of claim 1, wherein the nickel-titanium-based alloy is a nickel-titanium alloy.

5. The guide wire of claim 4, wherein an atomic percentage of nickel of the nickel-titanium alloy is between 49% and 53%.

6. The guide wire of claim 1, wherein:
the nickel-titanium-based alloy is a nickel-titanium-copper alloy and the nickel alloy is a nickel-copper alloy; or
the nickel-titanium-based alloy is a nickel-titanium-niobium alloy and the nickel alloy is a nickel-niobium alloy.

7. The guide wire of claim 1, wherein the core shaft comprises:
a small diameter portion defining a distal end of the core shaft;
a large diameter portion having an outer diameter that is larger than an outer diameter of the small diameter portion; and
a tapered portion extending between the small diameter portion and the large diameter portion, an outer diameter of the tapered portion increasing in a direction from the small diameter portion to the large diameter portion.

8. The guide wire of claim 7, wherein:
the small diameter portion and the large diameter portion are each cylindrical; and
the tapered portion is frustoconical.

9. The guide wire of claim 7, wherein the small diameter portion includes each of the layered portion(s).

10. The guide wire of claim 9, further comprising a coil disposed around at least the small diameter portion of the core shaft.

11. The guide wire of claim 10, wherein the coil comprises a stainless steel, a superelastic alloy, and/or a radiopaque metal.

12. The guide wire of claim 10, wherein the coil comprises a wire having a diameter that is between 0.01 and 0.10 millimeters.

13. The guide wire of claim 10, further comprising:
a distal end fixing part that defines a distal end of the guide wire;
wherein the distal end of the core shaft and a distal end of the coil are each fixed to the distal end fixing part.

14. The guide wire of claim 13, wherein the distal end fixing part is hemispherical.

15. The guide wire of claim 13, wherein the distal end fixing part comprises an Sn—Pb alloy, an Pb—Ag alloy, an Sn—Ag alloy, and/or an Au—Sn alloy.

16. The guide wire of claim 10, wherein a proximal end of the coil is fixed to the tapered portion of the core shaft.

17. The guidewire of claim 7, wherein:
an axial length of the small diameter portion is between 0.5 and 50 mm; and
the outer diameter of the small diameter portion is between 0.02 and 0.1 mm.

18. The guidewire of claim 7, wherein:
an axial length of the tapered portion is between 10 and 200 mm;
the outer diameter of the tapered portion at the small diameter portion is between 0.02 and 0.1 mm; and
the outer diameter of the tapered portion at the large diameter portion is between 0.25 and 1 mm.

19. The guidewire of claim 7, wherein the outer diameter of the large diameter portion is between 0.25 and 1 mm.

20. The guidewire of claim 1, wherein a total length of the core shaft is between 1,800 and 3,000 mm.
